(19) United States
(12) Patent Application Publication
(10) Pub. No.: US 2004/0185430 A1
(43) Pub. Date: Sep. 23, 2004

(54) Process for evaluating the cosmetic or dermatological activity of a product and use of plant material

(76) Inventors: Marc Andre Lefebvre, Coulombiers (FR); Richard Martin, Rochecorbon (FR); Myriam Kauffmann, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/742,192

(22) Filed: Dec. 22, 2003

Related U.S. Application Data
(60) Provisional application No. 60/441,318, filed on Jan. 22, 2003.

(30) Foreign Application Priority Data
Dec. 20, 2002 (FR) 02 16390

(51) Int. Cl. C12Q 1/00

(52) U.S. Cl. 435/4

(57) ABSTRACT

Disclosed herein is a process for evaluating the cosmetic or dermatological activity of a product, comprising bringing the product to be evaluated into contact with a plant material and determining the variation induced by the product to be evaluated in at least one parameter chosen from mechanical, optical, physical, and chemical parameters of the plant material in the region of contact with the product as well as a process for screening activity principles, a method for marketing a product, a process for identifying undesirable effects of multiple active principles, and a combination comprising a cosmetic product and a notification indicating that the cosmetic process has been evaluated by using a plant material.

This application claims benefit of U.S. Provisional Application No. 60/441,318, filed Jan. 22, 2003.

The present disclosure relates to processes for evaluating or screening the activity of a product in the cosmetics or dermatological fields and to the use of a novel model for evaluating the cosmetic or dermatological activity, such as the effectiveness and/or the organoleptic properties, of such a product as more specifically described herein.

In determining the activity of a molecule or of a product, or selecting active molecules or products from a group of potential candidates, the use of in vivo tests is usually the route, which can give the results that can be best extrapolated to a large-scale application in man. However, this type of experiment cannot be carried out on human beings in the first place, since the test product has to have been recognized as being without danger, both toxicologically and immunologically. Further, even if they could be performed, clinical trials additionally involves statutory precautions and informing the subjects, which can slow down its implementation. Animal experimentation, sometimes used as an alternative, cannot be substituted for ethical reasons. In addition, the cosmetic target cannot always be applied in this model, for example, if it concerns a perception.

In vitro tests are conventionally used to objectively determine the activity of a novel product or a novel activity of a known product. They generally relate to cell cultures which can mimic mechanisms responsible in vivo, for the targeted activities. The extrapolation to man of these results can sometimes be difficult. Furthermore, their analysis may require highly effective and very sensitive techniques when it is desired to objectively determine cosmetic effects, the activity threshold of which can be low, or the appearance of the consequences is slow.

Models on reconstructed skin can give good results; however, they relate to techniques which can be problematic to employ.

It would therefore be desirable to have available an early evaluation on a living model which can be standardized and which does not involve the constraints of a toxicological or clinical phase. It would also be desirable to have available a method which can complement the in vitro and in vivo approaches in man and which can make it possible to better predict the intrinsic in vivo activities of the products or active principles envisaged.

Unexpectedly, the present inventors have been able to show that at least one of the above-mentioned desirables can be achieved by the use of a plant model according to the disclosure.

Disclosed herein is thus a process for evaluating the cosmetic or dermatological activity of a product, comprising bringing the product to be evaluated into contact with a plant material and determining the variation induced by the product to be evaluated in at least one parameter chosen from mechanical, behavioural, optical and physical and chemical parameters of the plant material. The parameter can also be chosen from biological parameters.

The variation induced by the product can be determined according to the disclosure in at least one of the following regions: the region of contact of the plant material with the product to be evaluated, a region distant from the region of contact, and a region distant from the region of application of the product.

Because plants are living organisms of very great diversity, plants can mimic a large number of conditions of the skin or superficial body growths. The plant material can, for example, act as a model for evaluating the cosmetic or dermatological activity chosen from the effects on surface conditions, for example, of the skin, and on internal metabolic reactions, such as enzymatic reactions, secretory activities and gas exchanges.

The plant can thus reproduce, for example, the defence mechanisms deployed by the skin, whether they concern the barrier effect, moisturizing or desquamation, or the combating of oxidation, pollution or light radiation. The plant can also mimic the transmission of information of a sensitive nature.

As disclosed herein, the term “product” means both an ingredient or an active principle in a more or less purified form, exhibiting an intrinsic in vitro or in vivo activity, and a formulation comprising at least one of these ingredients and at least one carrier such as at least one adjuvant suited to the targeted application.

This process disclosed herein can, for example, be suited to the evaluation of cosmetic products.

The term “cosmetic product” means, for example, any substance or preparation intended to be brought into contact with the various surface parts of the human body (epidermis, body hair and head hair, nails, lips and external genitals) or with the teeth and the buccal mucous membranes for the purpose, solely or mainly, of cleaning them, of fragrancing them, of modifying the appearance thereof, of correcting body odours, of protecting them, and/or of keeping them in good condition (Cosmetics Directive 76/768/EEC, amended).

In addition, as disclosed herein, the term “cosmetic product” also means active principles or compositions intended to be absorbed by any route which makes possible systemic passage, such as by the oral route, for the purpose of protecting or keeping in good condition the surface parts of the body or of improving the appearance of individuals, for example, with regard to the skin and its appendages.

Mention may be made, as non-limiting examples of cosmetic products, of any product intended to reduce signs of ageing of the skin and hair, such as wrinkles, to moisturize the skin, to clean, to nourish and/or to maintain the skin and hair, to deodorize the skin, to prepare it for exposure to sunlight, to reinforce the elasticity thereof, to improve the softness thereof, and the like.

The cosmetic or dermatological activities which can be evaluated according to the disclosure can be chosen, for example, from effects on the moisturizing of the skin, antiwrinkle effects, improvements in the barrier function, protection with regard to solar radiation, improvements in the mechanical properties of the skin, tensor effects, desquamation, pigmentation, for example, of the hair and the skin, effects of interaction with odours or their modification, such as weakening, transformation and capture, for example, the deodorizing or deodorant effects but also the effects of enhancing fragrance, soothing and anti-irritant effects, covering properties, colouring and masking optical properties, and hold of the product.

The present inventors have shown that this evaluation process can be carried out in a reproducible way with a good predictive factor. Furthermore, the plant material is a living organism, having its physiology, and it is subject to the environment, like the cutaneous covering or appendages in man, which can make it possible to reproduce several conditions to which a human body will be subjected during the application of the test products.

The plant material useful for the invention are advantageously issued from a superior organism, with eucaryotic cells. A photosynthetic organism, for example, can be used. Parameters which will be measured on such plant material according to the invention will not be the result of genetic modifications, but will be present in the natural plant used for the process, whether cultivated in vivo or in vitro, as explained below.

The process according to the disclosure can be carried out on a plant organism in its entirety or on fragments thereof. Use may, for example, be made, according to the disclosure, of the entire plant, flowers, leaves, petals, stems, fruits, seeds or roots, and/or the fragments thereof. In the case of a fruit, it is possible, for the evaluation of certain properties, to remove the waxy coating present on some varieties or to remove the skin, in order to promote gas exchanges.

The plant material can result from in vivo or in vitro cultivation. The term “in vivo cultivation” means any cultivation of conventional type, i.e., in the soil, in the open air or under glass, or alternatively soilless.

The term “in vitro cultivation” means all the techniques known to a person skilled in the art which can make it possible to artificially produce a plant or a part of a plant. The selection pressure imposed by the physicochemical conditions during the growth of plant cells in vitro can make it possible to obtain a standardized plant material which can be available throughout the year, in contrast to plants cultivated in vivo.

In one embodiment, the plant material is produced from dedifferentiated cells or from plantlets obtained by in vitro micropropagation.

The term “dedifferentiated plant cells” means any plant cell not exhibiting any of the characteristics of a specific specialization and capable of living by itself and not in dependence with other cells. These dedifferentiated plant cells are optionally capable, under the effect of an induction, of any differentiation in accordance with their genome (somatic embryos, root cultures, and the like).

Depending on the method of culturing chosen, and, for example, depending on the culture medium chosen, it is possible to obtain, from the same explant, dedifferentiated plant cells exhibiting different characters.

In one embodiment, the product to be evaluated is brought into contact with at least one part of a plant cultivated in vivo obtained from a plant material which has been subjected to in vitro culturing or propagation.

Contact can be achieved by topical application of the test product. This application to at least one portion of the external surface of the plant can be carried out by any methods known to a person skilled in the art suited to the form of the test product, such as spreading, spraying, steeping, injection, scarification and bringing into contact under occlusion.

In one embodiment, a part of the plant material is treated with the test product, optionally in an excipient; another part of the same plant material is not treated, or is treated only with the excipient, and can act as control for the determination of the variation.

In another embodiment, the product is brought into contact with the plant material in the form of a nutriment which can be absorbed by the plant. The plants grow on nutrient media supplemented by the test active principle, which is incorporated via the systemic route. The effects on the external layers of the plant can be evaluated as above, then mimicking the phenomena observed in oral cosmetics. Active principles which can, for example, be evaluated by this route are chosen from vitamins, trace elements, and components which act on the microbial flora.

In all cases, the application may be a single application or may be repeated over time, for example, at regular intervals for several hours, several days or several weeks.

In one embodiment, the conditions to which the plant material is subjected are controlled and can be subjected to modifications. These conditions include environmental parameters, such as luminosity, solar radiation, temperature, gas or free radical composition, the presence of contaminating particles, and relative humidity.

Various factors, such as stress, pollution and exposure to the sun, which can influence the cosmetic characteristics of a product or which are precisely those with respect to which the effectiveness of the product has to be evaluated, can thus be mimicked.

Any plant or plant fragment can be used in the process according to the disclosure. For example, the choice of the model can be suited to the cosmetic or dermatological activity which it is desired to evaluate. Mention may be made, without implied limitation, of the plants of the family of the Cactaceae and generally of desert and halophilic plants, which can, for example, be suited to the study of hydric stress. Plants of high mountains, such as Leontopodium alpinum, can, for example, be used to evaluate an activity with respect to UV stress; red fruits can also be used to evaluate a protective effect with regard to UV radiation. Models suited to measuring the moisturizing or the barrier effect of a product can, for example, be chosen from tubers, fruits and entire plants, among which may be mentioned, without implied limitation, Solanum tuberosum, Malus sp., Daucus carota, Ficus benjamina, Syngonium sp. and Spatyphillum sp. Further, the effect of U.V. radiation with or without application of UV screen can be evaluated on a plant material by, for example, Raman spectroscopy, even if the chlorophyll has inherent fluorescent properties.

The variation in the mechanical, optical and/or physicochemical parameter, for example, biochemical parameter, can be determined according to the disclosure by any qualitative or quantitative method known to a person skilled in the art.

The plant material can, for example, be observed by optical or spectroscopic means, evaluated by instrumental means, and/or analysed with the same techniques as those provided in bioanalysis for taking samples of stratum corneum, of mucous membrane, of hair cuticle, of nail surface, or of the secretions which cover them.

The variation in at least one parameter chosen from the optical, behaviour, biological, mechanical and physical and chemical parameters can be determined, for example, using the techniques conventionally used in cosmetics evaluation: corneometer, torquemeter, colorimeter, pH measurement, sebumeter, lipometer, laser and white-light confocal microscopes, desquamation, thickness of the corneocyte envelope, microcirculation, and optionally by measurements of ions or of electric potentials, indeed even medical imaging techniques, such as echography, MRI, and the like), insensible water loss, measurement of cutaneous folding (DensiScore), optical and photon microscopic observations, photographing, measurement of spectra (UV, visible, IR, Raman, fluorescence), such as spectrophotometry and spectroradiometry, chromatographic analysis of representative samples, such as gas chromatography, impression on organic materials (Silflo) and inorganic materials (ceramic).

The physical, chemical, and biological parameters can generally be measured by any appropriate assay method known to a person skilled in the art or by any genomic, transcriptomic or proteomic technique.

In one embodiment, the parameter measured or evaluated for the plant material is the same as that which would mark the activity of the product in man. This is because, in a number of cases, after contact with the product and/or exposure to environmental conditions, a similar, although different in magnitude, change in the mechanical and/or physicochemical factors can be observed in man and in the plant.

In another embodiment, the parameter measured or evaluated for the plant material is different from that which would mark the activity of the product in man, but its change can be correlated with an activity established in man. In this respect, mention may be made of, for example, mechanisms of reaction to various kinds of light radiation.

The simple observation of the appearance of the plant material can make possible access to parameters such as radiance and good health and can make it possible to draw up atlases, which can make it possible to improve the measurement of these parameters of “clinical” type.

The various optical, photography, and video observations can demonstrate the variations in the surface microrelief.

The moisturizing parameters can be measured in a way known per se using a corneometer.

The mechanical properties of the protective surface, such as the firmness or elasticity, essential to the role of the stratum corneum can be determined, for example, in the same way on the plant material by dermal torquemetry.

Colorimetry can make it possible to finely evaluate the modifications in colour of the plant material and the protective effects of a product, such as sunscreens and antioxidants; conversely, this determination can make it possible to assess the desquamating power of an active principle or the detergent power of a toilet product.

This technique can also be used to assess the cosmetic properties of make-up products, such as a foundation, nail varnish, lipstick, mascaras, and a hairstyling product.

In one embodiment, seeds are used as a model, such as horse chestnuts resulting from Aesculus hyppocastanum. This model can, for example, be suited to the study of products applied to the surface of the nail, for which it can mimic:

the domed shape,

the texture of its surface, which is glossy, close to the natural glossiness of the nail (and thus to its surface condition in terms of microrelief and of porosity), on the brown part, the whitish part (the hilum), which is less glossy, mimicking the surface condition of the nails which are naturally less glossy or after abrasion by a nail file or microbuffing by a manicuring instrument,

its colour: the rounded light part resembles the lunula and the change from the light part to the dark brown part, rounded in the convex direction, mimics the change from the lunula to the ungual plate. The beige/brown change is also equivalent to that of the plate at the free edge of the nail in terms of contrast in colours, although the concavity is reversed.

These colours can, for example, be close to those of the nails of black or dark-complexioned subjects.

the heterogeneities in the relief: the central beige part is often incised by a few hundred micrometres, which is the same order of magnitude as the irregularities observable on healthy nails (such as the furrows in aged nails or a mark of a break in the growth rhythm).

Models of sweet chestnuts, fruits of Castanea sativa, which can reproduce these furrows, can also be well suited to this type of evaluation.

These seeds are therefore substrates which can be very easy to employ and which can be highly representative in testing the optical properties of nail varnishes (the make-up result in terms of colour, 3D highlights on a domed surface, glossiness on a relatively matt organic substrate, coverage of the colour, coverage of the defects or of the heterogeneities of the nails), which can be the main purpose of make-up varnishes.

It is also possible to study the effect of a base (for example, smoothing base or concealing base) or of the superimposition of several coats.

The application of varnish can be easy and fast. The varnish can hold well. It can be removed with a solvent for nail varnishes and reapplied.

The general effect can be, for example, advantageous in judging the making-up effect of the formulations on the nails of dark-complexioned subjects.

The secretions at the surface of the leaves and/or fruits can be measured by sebumetry or lipometry as indicator of an effect on the secretion of sebum in man. Confocal measurements can be carried out in white light or with a laser to evaluate the condition of layers analogous to those of the stratum corneum.

The desquamation can be measured by collecting surface components which can spontaneously detach or by calibrated scraping of the plant and comparing their amount with that of a control plant.

The variations in thickness of different protective layers can thus be measured ultrasonically.

The effects on the microcirculation can be evaluated by monitoring the plant fluids which can roughly mimic the lymph and the blood, which are the phloem (elaborated sap) and the xylem (crude sap). The xylem moves from the roots to the apex, whereas the phloem moves in the opposite direction.

In one embodiment, the evaluation of the activity of a product with regard to the barrier effect comprises:

a) applying the product to be evaluated to a portion of plant material;

b) keeping contact for a period of time ranging from 1 to 10 hours;

c) measuring the insensible water loss (i) on the portion of plant material which has been in contact with the product to be evaluated and (ii) on another portion of the same plant material which has not been in contact with the product; and

d) comparing the results of the measurements on the two portions.

The sample can be taken by any method known to a person skilled in the art, such as by the use of the device as disclosed in document FR-A-2 667 778, which device can make it possible to circulate a liquid over the skin for the purpose of recovering components present on the surface of the skin. Use may also be made of the devices disclosed in document FR-A-2 368 708 and U.S. Pat. No. 5,433,214.

The taking of the sample can, for example, be carried out by a device for measuring and/or analysing at least one parameter chosen, for example, from biological, mechanical, chemical and physical parameters, of an external portion of the human body, such as the skin, nails and hair, as disclosed in document FR 0 114 334. This device comprises a surface intended to be placed in a position facing or in contact with the external portion, wherein the device is:

either able to be modelled to the external portion and comprising at least one inorganic material;

or unable to be modelled to the external portion and made essentially of at least one inorganic material, wherein the surface comprises at least one inorganic material, other than glass, when it is not in the fibrous or particulate form.

Such a device can be used to determine the condition of the stratum corneum, to analyse the secretions, excretions or odours present at the surface of the plant material, or to analyse its bioanalytical, microbiological (bacteria, viruses, yeasts, acarids, various parasites and the like) or enzymatic content.

The components which can be sampled by the device according to the disclosure can be in the solid, liquid or gas (volatile and/or odoriferous compounds) form.

Such a device can also be used for evaluation of mechanical properties of a coating, such as its elasticity, or physical properties of a surface, such as its relief or microrelief, the number and/or the depth of the wrinkles or microwrinkles or grooves present at its surface, by the density and/or the size of its pores or ostia, or the smoothness or the slipperiness.

The measurement of the variation in a chemical parameter can make it possible, for example, to determine the appearance or the modification of a biological or biochemical reaction by the active principle or the product to be evaluated. It can thus be possible to measure the appearance of at least one metabolite or the variations in the concentrations of biological constituents, substrates or cosubstrates of the biological reactions. These assays can relate, for example, to enzymatic reactions and can, for example, be carried out by at least one method chosen from those enzymatic, immuno-enzymatic and histochemical method known to a person skilled in the art.

In one embodiment, the process is suited to the evaluation of the protective or activating effect of the test product on a metabolic reaction, under conditions or in the presence of a factor which is known to influence the metabolic reaction.

The analysis of the gases in a closed chamber or a chamber in contact with the surface of the plant, such as the modifications in at least one of the O₂, CO₂ and H₂O₂ contents, carried out, for example, by chromatography, can make it possible to study the cell metabolism such as the modifications, symmetrical to those observed for the cutaneous metabolism during irradiation or for the evaluation of agents for combating pollution or free radicals.

For example, the emission of odour into the environment can be monitored by direct gas chromatography techniques on the plant such as the head space technique, which can make it possible to collect the most volatile molecules, which would be undetectable with regard to stored samples. By way of example, the studies can be carried out directly on onions (Alium cepa), garlic (Alium sativum), Euphorbia sp. or other parts of food or aromatic plants, for which a part of the molecules emitted is similar to those which are excreted in human sweat. It is possible to interpose, by application to or spraying over the plant, odour-capturing systems, such as cyclodextrins, zeolites or various chemical catalysts, in order to evaluate their effects.

It is also possible to evaluate, for pleasant smells, such as those of flowers and of fruits, the effect of compositions intended to enhance these smells or to prolong their presence close to the integuments.

The evaluation process according to the disclosure can, for example, be used to screen multiple active principle. In one embodiment, disclosed herein is a screening process for identifying cosmetic or dermatological active principles, comprising

a) evaluating the cosmetic or dermatological activity of the multiple active principles by the process as defined above in paragraph [060],

b) comparing the variations induced by the multiple active principles to be screened in at least one parameter chosen from mechanical, behaviour, biological, optical, physical, and chemical parameters of the plant material, and

c) selecting one or any combination of active principles which induced the most significant variations in b), and optionally, selecting at least one active principle that induced less than the most significant variation in b).

The active principle or principles thus selected can, for example, be incorporated in a formulation, the composition of which can thus be validated. Further disclosed herein is therefore a process for preparing a cosmetic or dermatological composition, comprising

i) selecting one or any combination of active principles by the process defined above in paragraph [071], and

ii) mixing at least one of the active principles selected in i) with at least one carrier chosen from physiologically and cosmetologically acceptable carriers.

Further disclosed herein is thus the use of at least one plant material or of at least one fragment of the at least one plant material to evaluate the cosmetic or dermatological activity of a product.

Further disclosed herein is the use of a plant model onto which the consumers, such as female consumers, can be projected. Disclosed herein is thus a method for marketing a product, such as a cosmetic product, comprising performing the evaluation process of the product as discussed above in paragraph [060], and

providing a notification indicating that the product has been evaluated by using a plant material. The evaluation process can be based on the specific plant model of at least one of the product and a product line.

Further disclosed herein is a method for marketing a cosmetic product, comprising providing a notification indicating that a cosmetic product has been evaluated by using a plant material. Even further disclosed herein is a combination comprising a cosmetic product and a notification indicating that the cosmetic product has been evaluated by using a plant material.

The notification can indicate that the cosmetic product has been evaluated by placing the cosmetic product in contact with the plant material. The notification can also indicate that at least one of an efficacy, an effect, and a property of the cosmetic product has been evaluated using the plant material. Further, the notification can indicate that the cosmetic product has been evaluated using a plant model associated with at least one of the cosmetic product and a cosmetic product line.

The plant model according to the disclosure can make it possible to more clearly explain the mechanisms to non-specialists in the field under consideration and, for example, to make children understand why they should not expose themselves too much to the sun.

The notification disclosed herein can be carried out by any channel of communication. It can be carried out, for example, by a statement by an individual, such as a salesperson, directly at the point of sale, or by radio and television, such as in the context of commercials. It can also be carried out by broadcasted advertisement. The broadcasted advertisement can be chosen, for example, from an electronic mail, a website page, a web cast, an audio broadcast, an analog video broadcast, a digital video broadcast, a television broadcast, a radio broadcast, a broadcast via a telephone network, a broadcast via a computer network, and a broadcast via the Internet. The indication can also be carried out by a printed material associated with the product, chosen, for example, from printed material appearing on packaging associated with the product, printed material contained in packaging associated with the product, a product label, a display at a point of sale for the product, an advertisement, a sign, a mailing, and a flyer.

Visual images can also be used to carry out the notification in association with the marketing methods disclosed herein, such as in the form of photographs, videos or any other form of illustration, such as graphics. Such visual images can also be representative of the effect of the product on the plant material.

In one embodiment, the activity evaluated by the process according to the disclosure can be an undesirable activity, such as a toxicity or an irritant effect of the product for the skin, mucous membranes and/or superficial body growths, indeed even for the entire body. Further disclosed herein is thus a screening process for identifying undesirable effects of multiple active principles chosen from cosmetic and dermatological active principles, comprising:

a) bringing the multiple active principles into contact with a plant material;

b) determining the variations induced by the multiple active principles in at least one parameter chosen from mechanical, behaviour, biological, optical, physical, and chemical parameters of said plant material, wherein the at least one parameter is associated with an undesirable effect of the multiple active principles on a biological marker in a normal condition;

c) comparing the variations induced by the multiple active principles in at least one parameter chosen from the mechanical, behaviour, biological, optical, physical, and chemical parameters of the plant material; and

d) selecting one or any combination of active principles which induced the least significant variation in c), and optionally, selecting at least one active principle that induced more than the least significant variation in c).

This process can, for example, substitute for the Ames test.

Other advantages of the disclosure will become apparent on reading the following non-limiting examples.

### EXAMPLE 1

### Evaluation of the Moisturizing Effect

I. Material and Method

Plant Model and Human Model

1) The plants used were as follows:

Moisturizing and Barrier Effect:

Potatoes, BF15 (market garden origin)

Carrots (market garden origin)

Apples, Golden Delicious (market garden origin)

Ficus (in pots sheltered from direct sunlight)

Syngonium (in pots sheltered from direct sunlight)

Spatyphyllum (in pots sheltered from direct sunlight)

The plants were placed in a controlled environment in order to carry out the measurements and tests.

2) To measure the moisturizing and the barrier effects, the human models (2 male volunteers and 2 female volunteers) took part in the tests and measurements under environmental conditions which were also controlled.

Cosmetic Active Principles and Products Used:

Moisturizing and Barrier Effect:

Petroleum jelly

3% Glycerol solution

13% Lauryl phosphate (surfactant)

Aquasource® (Biotherm)

Effacil® (Lancome)

Excell A3®

Protocol of Measurements

Measurements were carried out at approximately 22° C. and a relative humidity ranging from 53% to 67%.

Applications on Plant Model or on Human Model:

Fruits and vegetables: 2 samples per product, measured at intervals of 24 hours. Application to regions of 4 cm². A control region on each sample.

Plants: 2 samples per product, measured at intervals of 24 hours. Application of the formulations over the whole of the vernicose (upper) surface. One leaf per formulation+one control leaf. The leaves chosen had a comparable appearance.

Human beings: 4 models per product. Application to an inner region of the arm. Three regions of measurements, including a control, on the inner part of each forearm were conducted. The models were kept calm under climate-controlled measuring conditions for 15 minutes before each series of measurements. Application of each formulation over an area of 16 cm².

Application of the formulations at the rate of 4 mg/cm² (search for the direction of the action and not for the performance of the product).

Measurements of the moisturizing and of the barrier effects (Plant Model and Human Model)

Moisturizing—Measurement with a CM820 corneometer of the mean value of 5 determinations.

Measurements at:

T0: reference value before application of the formulations.

T 2 hours for the active principles (single measurement after observation of appearance of cuts on the leaves brought about by the measuring pressure of the corneometer).

T 1 hour and T 4 hours for the commercial formulations.

II. Results

Results of the Measurements of the Moisturizing (Plant Model and Human Model)

The values are the mean of the measurements at Ti divided by those at TO corrected for the variations in the control regions. The agreement between man and plant was obtained by the comparable change in the measurements.

| | Variation |
|---|---|
| | T 2 hours/T0 |
| Glycerol | Corneo |
| Carrot | 62% |
| Potato, BF15 | 69% |
| Apples, Golden Delicious | 16% |
| Ficus | 47% |
| Syngonium | 38% |
| Spatyphillum | 52% |
| Man | 22% |

| | Variation |
|---|---|
| | T 2 hours/T0 |
| Petroleum jelly | Corneo |
| Carrot | 80% |
| Potato, BF15 | 9% |
| Ficus | 44% |
| Syngonium | 48% |
| Spatyphillum | 35% |
| Man | 12% |

| | Variation |
|---|---|
| | T 2 hours/T0 |
| Surfactant | Corneo |
| Carrot | 271% |
| Potato, BF15 | 1144% |
| Apples, Golden Delicious | 2% |
| Ficus | 20% |
| Syngonium | 48% |
| Spatyphillum | 30% |
| Man | 5% |

| | Variation |
|---|---|
| | T 1 hour/T0 |
| Aquasource | Corneo |
| Carrot | 33% |
| Potato, BF15 | 55% |
| Apples, Golden Delicious | 65% |
| Ficus | 150% |
| Syngonium | 100% |
| Spatyphillum | 193% |
| Man | 21% |

| | Variation |
|---|---|
| | T 1 hour/T0 |
| Effacil | Corneo |
| Potato, BF15 | 24% |
| Apples, Golden Delicious | 67% |
| Spatyphillum | 56% |
| Man | 2% |

| | Variation |
|---|---|
| | T 1 hour/T0 |
| Excell A3 | Corneo |
| Carrot | 44% |
| Potato, BF15 | 67% |
| Apples, Golden Delicious | 78% |
| Syngonium | 89% |
| Spatyphillum | 21% |
| Man | 13% |

### EXAMPLE 2

### Evaluation of the Barrier Effect

The plant and human models and the products tested were the same as in Example 1.

The IWL (insensible water loss) measurements were carried out according to the same protocol as above.

IWL: Measurement with a Dermalab (vertical probe in contact with the measurement region, under minimum pressure). For the plants, a glass sheet was placed under the measurement region. 2 series of consecutive measurements of 25 values were conducted.

The values are the mean of the measurements at Ti divided by those at T0 corrected for the variations in the control regions. The agreement between man and plant was obtained by the comparable change in the measurements.

| | Variation |
|---|---|
| | T 2 hours/T0 |
| Glycerol | IWL |
| Carrot | 1% |
| Potato, BF15 | 188% |
| Ficus | 183% |
| Syngonium | 40% |
| Spatyphillum | 14% |
| Man | 55% |

| | Variation |
|---|---|
| | T 2 hours/T0 |
| Petroleum jelly | IWL |
| Carrot | −26% |
| Potato, BF15 | −28% |
| Apples, Golden Delicious | −42% |
| Ficus | −25% |
| Syngonium | −72% |
| Man | −24% |

| | Variation |
|---|---|
| | T 2 hours/T0 |
| Surfactant | IWL |
| Carrot | 2% |
| Potato, BF15 | 48% |
| Apples, Golden Delicious | 0% |
| Syngonium | 104% |
| Spatyphillum | 13% |
| Man | 19% |

| | Variation |
|---|---|
| | T 1 hour/T0 |
| Aquasource | IWL |
| Carrot | 28% |
| Potato, BF15 | 1% |
| Syngonium | 2233% |
| Spatyphillum | 233% |
| Man | 19% |

| | Variation |
|---|---|
| | T 1 hour/T0 |
| Effacil | IWL |
| Carrot | 37% |
| Potato, BF15 | 339% |
| Ficus | 648% |
| Syngonium | 30% |
| Man | 13% |

| | Variation |
|---|---|
| | T 1 hour/T0 |
| Excell A3 | IWL |
| Carrot | −1% |
| Apples, Golden Delicious | −70% |
| Ficus | −23% |
| Syngonium | −7% |
| Spatyphillum | −93% |
| Man | −42% |

### EXAMPLE 3

### Evaluation of a Tensor Effect

Syngonium (in pots sheltered from direct sunlight) was used to evaluate the Tensor effect.

The plants were placed in a controlled environment in order to carry out the measurements and tests.

The composition T tested comprises 7% of a formulation based on rice starch 7% and on acrylamide/sodium 2-acrylamidoethylpropanesulphonate copolymer as an inverse emulsion at 40% in an isoparaffin/water mixture.

The measurement protocol was the same as that described in Example 1.

The tensor effect was measured using an extensometer (in vitro): Measurement according to the standard MTT procedure of the tensor effect of the composition. The samples were cut from Syngonium leaves. The results were compared with those obtained under the same conditions on isolated human horny layer (SC).

Results of the Measurements of the Tensor Effect (In Vitro)

| MTT | Isolated Human SC | Syngonium |
|---|---|---|
| Tensor Effect | −1.73 | −2.58 |
| Reliability Index | 0.33 | 0.32 |
| (95%) | | |

Observations: the Syngonium is twice as sensitive as the SC, with a better reproducibility and a greater ease of use.

Conclusion

The plant model, in the three activity targets, i.e., the moisturizing effect, the barrier effect, and the mechanical properties, is an advantageous model exhibiting a stronger dynamic than that of the human model. Thus, the values using a corneometer, of IWL and by extensometry change in the same direction as with regard to man.

What is claimed is:

1. A process for evaluating the cosmetic or dermatological activity of a product, comprising:
bringing the product to be evaluated into contact with a plant material; and
determining the variation induced by the product to be evaluated in at least one parameter chosen from mechanical, behaviour, biological, optical, physical, and chemical parameters of said plant material.

2. The process according to claim 1, wherein the variation induced by the product to be evaluated in at least one parameter chosen from mechanical, behaviour, biological, optical, physical, and chemical parameters of said plant material is determined in the region of contact of said plant material with the product to be evaluated.

3. The process according to claim 1, wherein the cosmetic or dermatological activity to be evaluated is chosen from the effects on surface condition, on secretions, on enzymatic reactions, and on gas exchanges.

4. The process according to claim 1, wherein the cosmetic or dermatological activity evaluated for the product is chosen from effects on the moisturizing of skin, antiwrinkle effects, improvements in the barrier function, protection with regard to solar radiation, improvements in the mechanical properties of the skin, desquamation, pigmentation, tensor effects, soothing and anti-irritant effects, effects of interaction with odours, covering properties, colouring and masking optical properties, and hold of the product.

5. The process according to claim 1, wherein the product to be evaluated is brought into contact with at least one part of a plant chosen from plants cultivated in vivo and cultivated in vitro.

6. The process according to claim 5, wherein the product to be evaluated is applied to, or close to, at least one portion of the external surface of the plant.

7. The process according to claim 5, wherein the product to be evaluated is brought into contact with the plant in the form of a nutriment that is absorbed by said plant.

8. The process according to claim 1, wherein the product to be evaluated is brought into contact with at least one part of a plant cultivated in vivo obtained from a plant material which has been subjected to in vitro culturing or propagation.

9. The process according to claim 1, wherein the Variation in at least one parameter chosen from mechanical, behaviour, biological, optical, physical, and chemical parameters is determined qualitatively.

10. The process according to claim 1, wherein the variation in at least one parameter chosen from mechanical, behaviour, biological, optical, physical, and chemical parameters is determined by at least one quantitative method.

11. The process according to claim 1, wherein the variation in at least one parameter chosen from mechanical, behaviour, biological, optical, physical, and chemical parameters is determined by at least one of the following techniques: corneometer, torquemeter, colorimeter, pH measurement, sebumeter, lipometer, laser and white-light confocal microscopes, desquamation, thickness of the corneocyte envelope, microcirculation, insensible water loss, measurement of cutaneous folding (DensiScore), optical and photon microscopic observations, photographing, measurement of spectra, chromatographic analysis, and impression on organic materials and inorganic materials.

12. The process according to claim 1, wherein the at least one parameter is chosen from physical and chemical parameters and the variation is determined by at least one enzymatic assay.

13. The process according to claim 1, wherein the plant material is chosen from entire plants, flowers, leaves, petals, stems, fruits, seeds, roots, and fragments thereof.

14. The process according to claim 1, wherein the product is chosen from cosmetic products.

15. A screening process for identifying cosmetic or dermatological active principles, comprising:
a) bringing multiple active principles to be screened into contact with a plant material;
b) determining the variations induced by the multiple active principles to be screened in at least one parameter chosen from mechanical, optical, physical, and chemical parameters of said plant material;
c) comparing the variations induced by the multiple active principles to be screened in at least one parameter chosen from mechanical, behaviour, biological, optical, physical, and chemical parameters of said plant material, and
d) selecting one or any combination of active principles that induced the most significant variation in c) and optionally, selecting at least one active principle that induced less than the most significant variation in c).

16. The screening process according to claim 15, wherein the variations induced by the multiple active principles to be screened in at least one parameter chosen from mechanical, behaviour, biological, optical, physical, and chemical parameters of said plant material are determined in the regions of contact of said plant material with the multiple active principles.

17. A process for preparing a cosmetic or dermatological composition, comprising:
i) bringing multiple cosmetic or dermatological active principles to be screened into contact with a plant material,
ii) determining the variations induced by the multiple active principles to be screened in at least one parameter chosen from mechanical, behaviour, biological, optical, physical, and chemical parameters of said plant material,
iii) comparing the variations induced by the active principles to be screened in the at least one parameter chosen from mechanical, behaviour, biological, optical, physical, and chemical parameters of said plant material,
iv) selecting one or any combinations of active principles that induced the most significant variation in iii) and optionally, selecting at least one active principle that induced less than the most significant variation in iii), and
v) mixing at least one of active principles selected in iv) with at least one carrier chosen from physiologically and cosmetologically acceptable carriers.

18. The process according to claim 17, wherein the variations induced by the active principles to be screened in at least one parameter chosen from mechanical, behaviour, biological, optical, physical, and chemical parameters of said plant material are determined in the region of contact of said plant material with the active principles to be screened.

19. A method of marketing a product, comprising:
performing the process of claim 1; and
providing a notification indicating that a product has been evaluated by using a plant material.

20. The method according to claim 19, wherein the notification indicates that the product has been evaluated by placing the product in contact with the plant material.

21. The method according to claim 19, wherein the notification indicates that variation induced by the product to be evaluated in at least one parameter chosen from mechanical, behaviour, biological, optical, physical, and chemical parameters of said plant material has been determined.

22. The method according to claim 19, wherein the notification indicates that the product has been evaluated using a plant model associated with at least one of the product and a product line.

23. The method according to claim 19, wherein the product is chosen from cosmetic products.

24. The method according to claim 19, wherein the notification comprises a statement by an individual.

25. The method according to claim 19, wherein the notification comprises a notification at a point of sale for the product.

26. The method according to claim 19, wherein the notification comprises a broadcasted advertisement.

27. The method according to claim 26, wherein the broadcasted advertisement comprises at least one of an electronic mail, a website page, a web cast, an audio broadcast, an analog video broadcast, a digital video broadcast, a television broadcast, a radio broadcast, a broadcast via a telephone network, a broadcast via a computer network, and a broadcast via the Internet.

28. The method according to claim 19, wherein the notification comprises a printed material associated with the product.

29. The method according to claim 28, wherein the printed material comprises at least one of printed material appearing on packaging associated with the product, printed material contained in packaging associated with the product, a product label, a display at a point of sale for the product, an advertisement, a sign, a mailing, and a flyer.

30. The method according to claim 19, wherein the notification comprises a visual image.

31. The method according to claim 30, wherein the visual image comprises at least one of an illustration, a photograph, a video, a graphic, a visual representation, and a symbol.

32. The method according to claim 30, wherein the visual image is representative of the effect of the product on the plant material.

33. A screening process for identifying undesirable effects of multiple active principles chosen from cosmetic and dermatological active principles, comprising:
a) bringing the multiple active principles into contact with a plant material;
b) determining the variations induced by the multiple active principles in at least one parameter chosen from mechanical, behaviour, biological, optical, physical, and chemical parameters of said plant material, wherein the at least one parameter is associated with an undesirable effect of the multiple active principles on a biological marker in a normal condition;
c) comparing the variations induced by the multiple active principles in at least one parameter chosen from the mechanical, behaviour, biological, optical, physical, and chemical parameters of the plant material; and
d) selecting one or any combination of active principles which induced the least significant variation in c), and optionally, selecting at least one active principle that induced more than the least significant variation in c).

34. A method of marketing a cosmetic product, comprising:
providing a notification indicating that the cosmetic product has been evaluated by using a plant material.

35. The method according to claim 34, wherein the notification indicates that the cosmetic product has been evaluated by placing the cosmetic product in contact with the plant material.

36. The method according to claim 34, wherein the notification indicates that at least one of an efficacy, an effect, and a property of the cosmetic product has been evaluated using the plant material.

37. The method according to claim 34, wherein the notification indicates that the cosmetic product has been evaluated using a plant model associated with at least one of the cosmetic product and a cosmetic product line.

38. The method according to claim 34, wherein the notification comprises a statement by an individual.

39. The method according to claim 34, wherein the notification comprises a notification at a point of sale for the cosmetic product.

40. The method according to claim 34, wherein the notification comprises a broadcasted advertisement.

41. The method according to claim 40, wherein the broadcasted advertisement comprises at least one of an electronic mail, a website page, a web cast, an audio broadcast, an analog video broadcast, a digital video broadcast, a television broadcast, a radio broadcast, a broadcast via a telephone network, a broadcast via a computer network, and a broadcast via the Internet.

42. The method according to claim 34, wherein the notification comprises a printed material associated with the product.

43. The method according to claim 42, wherein the printed material comprises at least one of printed material appearing on packaging associated with the product, printed material contained in packaging associated with the product, a product label, a display at a point of sale for the product, an advertisement, a sign, a mailing, and a flyer.

44. The method according to claim 34, wherein the notification comprises a visual image.

45. The method according to claim 44, wherein the visual image comprises at least one of an illustration, a photograph, a video, a graphic, a visual representation, and a symbol.

46. The method according to claim 44, wherein the visual image is representative of the effect of the cosmetic product on the plant material.

47. A combination comprising:
a cosmetic product; and
a notification indicating that the cosmetic product has been evaluated by using a plant material.

48. The combination according to claim 47, wherein the notification indicates that the cosmetic product has been evaluated by placing the cosmetic product in contact with the plant material.

49. The combination according to claim 47, wherein the notification indicates that at least one of an efficacy, an effect, and a property of the cosmetic product has been evaluated using the plant material.

50. The combination according to claim 47, wherein the notification indicates that the cosmetic product has been evaluated using a plant model associated with at least one of the cosmetic product and a cosmetic product line.

51. The combination according to claim. 47, wherein the notification comprises a statement by an individual.

52. The combination according to claim 47, wherein the notification comprises a notification at a point of sale for the cosmetic product.

53. The combination according to claim 47, wherein the notification comprises a broadcasted advertisement.

54. The combination according to claim 53, wherein the broadcasted advertisement comprises at least one of an electronic mail, a website page, a web cast, an audio broadcast, an analog video broadcast, a digital video broadcast, a television broadcast, a radio broadcast, a broadcast via a telephone network, a broadcast via a computer network, and a broadcast via the Internet.

55. The combination according to claim 47, wherein the notification comprises a printed material associated with the product.

56. The combination according to claim 55, wherein the printed material comprises at least one of printed material appearing on packaging associated with the product, printed material contained in packaging associated with the product, a product label, a display at a point of sale for the product, an advertisement, a sign, a mailing, and a flyer.

57. The combination according to claim 47, wherein the notification comprises a visual image.

58. The combination according to claim 57, wherein the visual image comprises at least one of an illustration, a photograph, a video, a graphic, a visual representation, and a symbol.

59. The combination according to claim 57, wherein the visual image is representative of the effect of the cosmetic product on the plant material.
